(19) United States
(12) Patent Application Publication
(10) Pub. No.: US 2015/0093768 A1
(43) Pub. Date: Apr. 2, 2015

(54) DETECTION OF PROSTATE AND BLADDER CANCER

(71) Applicant: Patricia A. KRUK, Tampa, FL (US)

(72) Inventor: Patricia A. KRUK, Tampa, FL (US)

(73) Assignee: UNIVERSITY OF SOUTH FLORIDA, Tampa, FL (US)

(21) Appl. No.: 14/505,123

(22) Filed: Oct. 2, 2014

Related U.S. Application Data
(60) Provisional application No. 61/885,742, filed on Oct. 2, 2013.

(51) Int. Cl. G01N 33/574

(52) U.S. Cl. CPC G01N 33/57434; G01N 33/57407; G01N 33/5748; G01N 2333/82
USPC 435/7.92; 435/287.2; 436/501; 422/69

(57) ABSTRACT

The present invention relates to a method for the diagnosis, prognosis, and monitoring of cancer, such as early or late stage prostate cancer and bladder cancer, in a subject by detecting Bcl-2 in a biological sample from the subject, preferably a urine or blood sample. Bcl-2 may be measured using an agent that detects or binds to Bcl-2 protein or an agent that detects or binds to encoding nucleic acids, such as antibodies specifically reactive with Bcl-2 protein or a portion thereof. The invention further relates to kits for carrying out the methods of the invention. The invention further relates to a device for the rapid detection of Bcl-2 in a bodily fluid and methods for rapidly measuring Bcl-2 in a bodily fluid.

### CROSS-REFERENCE TO RELATED APPLICATION

This application claims the benefit of U.S. Provisional Application Ser. No. 61/885,742, filed Oct. 2, 2013, which is hereby incorporated by reference in its entirety.

### GOVERNMENTAL SUPPORT

This invention was made with government support under Grant No. W81XWH-07-1-0276 awarded by the Department of Defense. The government has certain rights in the invention.

### BACKGROUND OF THE INVENTION

Prostate cancer is a leading cause of cancer death in men in the United States. Prostate cancer occurs when cells in the prostate grow and multiply uncontrollably, damaging surrounding tissue and interfering with the normal function of the prostate. Prostate cancer cells can spread to other parts of the body.

Early prostate cancer usually causes no symptoms. When prostate cancer does cause symptoms, they are often similar to those benign prostate diseases such as benign prostatic hyperplasia. Symptoms of prostate cancer include frequent urination, nocturia, difficulty starting and maintaining a steady stream of urine, hematuria, and dysuria.

Screening refers to looking for cancer in individuals who have no symptoms of the disease, while early detection is finding cancer at an early stage of the disease, when it is less likely to have spread (and is more likely to be treated effectively).

Early diagnosis of prostate cancer can significantly improve patient survival. Existing diagnostic methods for prostate cancer include physical examination, assaying biomarkers (such as prostate-specific antigen (PSA)), and biopsy. It would be advantageous to provide improved methods for diagnosis and prognosis prostate cancer.

While apoptosis is an essential biological process for normal development and maintenance of tissue homeostasis, it is also involved in a number of pathologic conditions including tissue injury, degenerative diseases, immunological diseases and cancer (Lowe, S. W. and Lin, A. W. Carcinogenesis, 2000, 21:485-495). Whether activated by membrane bound death receptors (Ashkenazi, A. et al. J. Clin. Invest., 1999, 104:155-162; Walczak, H. Krammer, P. H. Exp. Cell Res, 2000, 256:58-66) or by stress-induced mitochondrial perturbation with subsequent cytochrome c release (Loeffler, M. and Kroemer, G. Exp. Cell Res., 2000, 256:19-26; Wernig, F. and Xu, Q. Prog. Biophys. Mol. Biol., 2002, 78:105-137; Takano, T. et al. Antiox. Redox. Signal, 2002, 4:533-541), activation of downstream caspases leads to stepwise cellular destruction by disrupting the cytoskeleton, shutting down DNA replication and repair, degrading chromosomal DNA, and, finally, disintegrating the cell into apoptotic bodies (Nagata, S. Exp. Cell Res., 2000, 256:12-18). The key regulators of apoptosis include members of the bcl-2 protein family (Farrow, S. N. and Brown, R. Curr. Opin. Gen. Dev., 1996, 6:45-49).

The bcl-2 protein family consists of both pro- and anti-apoptotic protein family members that act at different levels of the apoptotic cascade to regulate apoptosis. The bcl-2 family members contain at least one Bcl-2-homology (BH) domain (Farrow, S. N. and Brown, R. Curr. Opin. Gen. Dev., 1996, 6:45-49). Though all bcl-2 family members demonstrate membrane channel forming activity, Bcl-2 (the archetypal bcl-2 family member) channels are cation (Ca⁺⁺) selective and, owing to its exclusive ER and mitochondrial membrane localization (Thomenius, M. J. and Distelhorst, C. W. J. Cell Sci., 2003, 116:4493-4499), the anti-apoptotic function of Bcl-2 is at least partly mediated by its ability to prevent calcium release from the ER and subsequent mitochondrial membrane perturbation and cytochrome c release.

### BRIEF SUMMARY OF THE INVENTION

The present invention relates to cancer screening. In one embodiment, the present invention relates to the use of Bcl-2 as a biomarker for prognosis, diagnosis, and monitoring of cancer, such as prostate cancer and bladder cancer. For example, Bcl-2 may be used to diagnose and monitor early stage and late stage prostate cancer. Bcl-2 may be used as a biomarker for prostate cancer and bladder cancer before surgery and after relapse. Bcl-2, and agents that bind Bcl-2 polynucleotides or polypeptides may be used to detect and monitor prostate cancer and bladder cancer.

Thus, more particularly, this invention relates to the detection of prostate cancer and/or bladder cancer by screening for elevated levels of Bcl-2 in biological samples, such as urine, blood (e.g., whole blood, serum, or plasma), and ascites fluid.

In one embodiment, if the subject's Bcl-2 level is higher than control (and thus indicates that the subject may have prostate cancer), the method further comprises one or more of the following steps: performing at least one additional diagnostic test or examination for prostate cancer (e.g., by assessing for the presence of one or more cancer symptoms, detecting additional cancer markers, detecting the presence of the cancer through an imaging modality such as X-ray, CT, nuclear imaging (PET and SPECT), ultrasound, MRI); informing the subject as having prostate cancer; and/or prescribing treatment for prostate cancer (e.g., by surgery, chemotherapy, and/or radiation).

In one embodiment, if the subject's Bcl-2 level is not higher than control (and thus indicates that the subject does not have prostate cancer), the method further comprises informing the subject as not having prostate cancer; and/or not prescribing treatment for prostate cancer. In another embodiment, if the subject's Bcl-2 level indicates that the subject does not have prostate cancer, the method further comprises performing at least one additional diagnostic test or examination for prostate cancer.

In another embodiment, if the subject's Bcl-2 level is higher than control (and thus indicates that the subject may have bladder cancer), the method further comprises one or more of the following steps: performing at least one additional diagnostic test or examination for bladder cancer (e.g., by assessing for the presence of one or more cancer symptoms, detecting additional cancer markers, detecting the presence of the cancer through an imaging modality such as X-ray, CT, nuclear imaging (PET and SPECT), ultrasound, MRI); informing the subject as having bladder cancer; and/or prescribing treatment of bladder cancer (e.g., by surgery, chemotherapy, and/or radiation).

In one embodiment, if the subject's Bcl-2 level is not higher than control (and thus indicates that the subject does not have bladder cancer), the method further comprises informing the subject as not having bladder cancer; and/or not prescribing treatment for bladder cancer. In another embodiment, if the subject's Bcl-2 level is not higher than control (and thus indicates that the subject does not have bladder cancer), the method further comprises performing at least one additional diagnostic test or examination for bladder cancer.

The invention also relates to kits for carrying out the methods of the invention.

In another aspect, the present invention relates to a device for the rapid detection of Bcl-2 in a bodily fluid such as blood or urine, for diagnosis and/or prognosis of prostate cancer and/or bladder cancer. Preferably, the device is a lateral flow device. In one embodiment, the device comprises an application zone for receiving a sample of bodily fluid such as blood or urine; a labeling zone containing a binding agent that binds to Bcl-2 in the sample; and a detection zone where Bcl-2-bound binding agent is retained to give a signal, wherein the signal given for a sample from a subject with a Bcl-2 level lower than a threshold concentration is different from the signal given for a sample from a patient with a Bcl-2 level equal to or greater than a threshold concentration.

In one embodiment, the test is a method for measuring Bcl-2 in a bodily fluid, comprising: (a) obtaining a sample of bodily fluid, such as blood or urine, from a subject; (b) contacting the sample with a binding agent that binds to any Bcl-2 in the sample; (c) separating Bcl-2-bound binding agent; (d) detecting a signal associated with the separated binding agent from (c); and (e) comparing the signal detected in step (d) with a reference signal which corresponds to the signal given by a sample from a subject with a Bcl-2 level equal to a threshold concentration. In one embodiment for diagnosis of prostate cancer and/or bladder cancer, the bodily fluid is urine, and the threshold urinary Bcl-2 concentration for the diagnosis of prostate and/or bladder cancer is 12 ng/ml or higher than 12 ng/ml.

### BRIEF DESCRIPTION OF THE DRAWINGS

FIG. 1 shows the results of a slot blot depicting urinary levels of Bcl-2. Urinary levels of Bcl-2 are higher in patients with ovarian cancer compared with normal healthy volunteers. Urine was collected from normal healthy volunteers and from patients with ovarian cancer. The urine was tested Bcl-2 by slot blot. Slot blots were performed using the Bio-Rad Bio-Dot SF® microfiltration apparatus according to the manufacturer's instructions. Membranes were incubated with anti Bcl-2 antibodies according to standard western blot procedure and visualized with enhanced chemiluminescence.

FIG. 2 is a histogram depicting urinary levels of Bcl-2 in normal and cancer patients. Additional urine specimens were collected from normal healthy volunteers and from patients with various cancers (including sarcoma, lung, brain, cervix, endometrial, colon, breast, melanoma, prostate, ovary, and bladder cancers). Slot blots were performed using the Bio-Rad Bio-Dot SF® microfiltration apparatus according to the manufacturer's instructions. Membranes were incubated with anti Bcl-2 antibodies according to standard western blot procedure and visualized with enhanced chemiluminescence. The data indicate elevated levels of Bcl-2 in the urine of patients with ovarian, prostate, and bladder cancer.

### BRIEF DESCRIPTION OF THE SEQUENCES

SEQ ID NO:1 is human Bcl-2 DNA (GenBank accession no. M14745); coding region (CDS): bases 32-751.

SEQ ID NO:2 is human Bcl-2 protein (GenBank accession no. AAA35591). SEQ ID NO:3 is human Bcl-2 DNA, transcript variant alpha (GenBank accession no. NM_{—}000633); CDS: bases 494-1213.

SEQ ID NO:4 is human Bcl-2 protein, transcript variant alpha (GenBank accession no. NP_{—}000624).

SEQ ID NO:5 is human Bcl-2 DNA, transcript variant beta (GenBank accession no. NM_{—}000657); CDS: bases 494-1111.

SEQ ID NO:6 is human Bcl-2 protein, transcript variant beta (GenBank accession no. NP_{—}000648).

### DETAILED DESCRIPTION OF THE INVENTION

The present invention relates to the use of Bcl-2 is an effective biomarker for the diagnosis, prognosis of cancer such as prostate and bladder cancer. Cancer markers (also called tumor markers) are molecules such as hormones, enzymes, and immunoglobulins found in the body that are associated with cancer and whose measurement or identification is useful in patient diagnosis or clinical management. They can be products of the cancer cells themselves, or of the body in response to cancer or other conditions. Most cancer markers are proteins. Some cancer markers are seen only in a single type of cancer, while others can be detected in several types of cancer. As with other cancer markers, Bcl-2 can be used for a variety of purposes, such as: screening a healthy population or a high risk population for the presence of cancer; making a diagnosis of cancer or of a specific type of cancer, such as prostate cancer and bladder cancer; determining the prognosis of a subject; and monitoring the course in a subject in remission or while receiving surgery, radiation, chemotherapy, or other cancer treatment.

In one embodiment, urine was collected from normal healthy volunteers (N=5) and from patients with cancer (N=5 per cancer type). Slot blots were performed using the Bio-Rad Bio-Dot SF® microfiltration apparatus according to the manufacturer's instructions. Membranes were incubated with anti Bcl-2 antibodies according to standard western blot procedure and visualized with enhanced chemiluminescence. The values are average densitometric values from 5 samples per cancer type. Urinary Bcl-2 was tested using slot blots with a Bcl-2 standard curve. The average amount of Bcl-2 in the urine of healthy volunteers was 11 ng/ml while that from pre-surgical patients with prostate cancer and bladder cancer averaged 15 ng/ml.

Taken together, these data indicate that quantification of urinary Bcl-2 protein appears to provide a novel, safe, sensitive, specific and economical method for the detection of prostate cancer and bladder cancer. Further, urinary levels of Bcl-2 can be used to monitor the presence of prostate cancer and bladder cancer and predict therapeutic and prognostic outcome.

In one embodiment, urinary Bcl-2 level can be used to distinguish subjects with prostate cancer from subjects with benign prostate tumor as well as normal, non-cancerous subjects. In one embodiment, urinary Bcl-2 level can be used to distinguish subjects with prostate cancer from subjects with benign prostate tumor as well as normal, non-cancerous subjects.

In one embodiment, urinary Bcl-2 level can be used to distinguish subjects with bladder cancer from subjects with benign bladder tumor as well as normal, non-cancerous subjects.

In one aspect, the invention includes a method for diagnosing cancer (such as, prostate cancer and bladder cancer) in a subject, comprising detecting the presence of Bcl-2 in a biological sample from the subject, such as urine, blood, peritoneal fluid, or ascites fluid, and wherein a level of Bcl-2 above a pre-determined threshold is indicative of cancer (such as prostate cancer and bladder cancer) in the subject.

In one embodiment of the method of the invention, the detecting comprises: (a) contacting the biological sample with a binding agent that binds Bcl-2 protein to form a complex; and (b) detecting the complex; and correlating the detected complex to the amount of Bcl-2 protein in the sample, wherein the presence of elevated Bcl-2 protein is indicative of cancer (such as, prostate cancer and bladder cancer). In a specific embodiment, the detecting of (b) further comprises linking or incorporating a label onto the agent, or using ELISA-based immunoenzymatic detection.

In some embodiments, the subject is suffering from cancer, such as prostate cancer and bladder cancer, and the detecting is performed at several time points at intervals, as part of a monitoring of the subject before, during, or after the treatment of the cancer.

Optionally, the methods of the invention further comprise comparing the level of Bcl-2 in the biological sample with the level of Bcl-2 present in a normal control sample, wherein a higher level of Bcl-2 in the biological sample as compared to the level in the normal control sample is indicative of cancer such as prostate cancer and bladder cancer.

In one embodiment, if the subject's Bcl-2 level is higher than control (and thus indicates that the subject may have prostate cancer), the method further comprises one or more of the following steps: performing at least one additional diagnostic test or examination for prostate cancer (e.g., by assessing for the presence of one or more cancer symptoms, detecting additional cancer markers, detecting the presence of the cancer through an imaging modality such as X-ray, CT, nuclear imaging (PET and SPECT), ultrasound, MRI); informing the subject as having prostate cancer; and/or prescribing treatment for prostate cancer (e.g., by surgery, chemotherapy, and/or radiation).

In one embodiment, if the subject's Bcl-2 level is not higher than control (and thus indicates that the subject does not have prostate cancer), the method further comprises informing the subject as not having prostate cancer; and/or not prescribing treatment for prostate cancer. In another embodiment, if the subject's Bcl-2 level indicates that the subject does not have prostate cancer, the method further comprises performing at least one additional diagnostic test or examination for prostate cancer.

In another embodiment, if the subject's Bcl-2 level is higher than control (and thus indicates that the subject may have bladder cancer), the method further comprises one or more of the following steps: performing at least one additional diagnostic test or examination for bladder cancer (e.g., by assessing for the presence of one or more cancer symptoms, detecting additional cancer markers, detecting the presence of the cancer through an imaging modality such as X-ray, CT, nuclear imaging (PET and SPECT), ultrasound, MRI); informing the subject as having bladder cancer; and/or prescribing treatment of bladder cancer (e.g., by surgery, chemotherapy, and/or radiation).

In one embodiment, if the subject's Bcl-2 level is not higher than control (and thus indicates that the subject does not have bladder cancer), the method further comprises informing the subject as not having bladder cancer; and/or not prescribing treatment for bladder cancer. In another embodiment, if the subject's Bcl-2 level is not higher than control (and thus indicates that the subject does not have bladder cancer), the method further comprises performing at least one additional diagnostic test or examination for bladder cancer.

In some embodiments, the subject exhibits no symptoms of prostate cancer and/or bladder cancer at the time the detecting of Bcl-2 is carried out. In other embodiments, the subject exhibits one or more symptoms of prostate cancer and/or bladder cancer at the time the detecting of Bcl-2 is carried out.

In another embodiment, the invention includes a method for prognostic evaluation of a subject having, or suspected of having, cancer, comprising: a) determining the level of Bcl-2 in a biological sample obtained from the subject, such as urine, blood, or ascites fluid; b) comparing the level determined in step (a) to a range of Bcl-2 known to be present in a biological sample obtained from a normal subject that does not have cancer; and c) determining the prognosis of the subject based on the comparison of step (b), wherein a high level of Bcl-2 in step (a) indicates an aggressive form of cancer and, therefore, a poor prognosis.

The terms “detecting” or “detect” include assaying or otherwise establishing the presence or absence of the target Bcl-2 (Bcl-2 encoding nucleic acid sequence or Bcl-2 gene product (polypeptide)), subunits thereof, or combinations of agent bound targets, and the like, or assaying for, interrogating, ascertaining, establishing, or otherwise determining one or more factual characteristics of gynecological cancer, metastasis, stage, or similar conditions. The detection of biomarker can be used for cancer diagnostic, prognostic, and monitoring applications. The term encompasses quantitative, semi-quantitative, and qualitative detection methodologies.

In embodiments of the invention involving detection of Bcl-2 protein (as opposed to nucleic acid molecules encoding Bcl-2 protein), the detection method is preferably an ELISA-based method. Preferably, in the various embodiments of the invention, the detection method provides an output (i.e., readout or signal) with information concerning the presence, absence, or amount of Bcl-2 in a sample from a subject. For example, the output may be qualitative (e.g., “positive” or “negative”), or quantitative (e.g., a concentration such as nanograms per milliliter).

In an embodiment, the invention relates to a method for detecting cancer in a subject by quantitating Bcl-2 protein in a biological sample such as urine from the subject, comprising (a) contacting (reacting) the biological sample with an antibody specific for Bcl-2 which is directly or indirectly labeled with a detectable substance; and (b) detecting the detectable substance.

In an embodiment, the invention relates to a method for diagnosing and/or monitoring cancer in a subject by quantitating Bcl-2 in a biological sample, such as urine or blood, from the subject, comprising (a) reacting the biological sample with an antibody specific for Bcl-2 which is directly or indirectly labeled with a delectable substance; and (b) detecting the detectable substance.

Embodiments of the methods of the invention involve (a) contacting a biological sample from a subject with an antibody specific for Bcl-2 which is directly or indirectly labeled with an enzyme; (b) adding a substrate for the enzyme wherein the substrate is selected so that the substrate, or a reaction product of the enzyme and substrate, forms fluorescent complexes; (c) quantitating Bcl-2 in the sample by measuring fluorescence of the fluorescent complexes; and (d) comparing the quantitated levels to that of a standard.

A preferred embodiment of the invention comprises the following steps:

(a) incubating a biological sample with a first antibody specific for Bcl-2 which is directly or indirectly labeled with a detectable substance, and a second antibody specific for Bcl-2 which is immobilized;

(b) separating the first antibody from the second antibody to provide a first antibody phase and a second antibody phase;

(c) detecting the detectable substance in the first or second antibody phase thereby quantitating Bcl-2 in the biological sample; and

(d) comparing the quantitated Bcl-2 with a standard.

A standard used in a method of the invention may correspond to Bcl-2 levels obtained for samples from healthy control subjects and/or from subjects with benign disease (such as benign prostate tumor, benign bladder tumor). Increased levels of Bcl-2 as compared to the standard may be indicative of cancer, such as early or late stage prostate cancer.

The invention also contemplates using the methods, devices, and kits described herein in conjunction with one or more additional markers (“biomarkers”) for cancer. Therefore, the invention contemplates a method for analyzing a biological sample for the presence of Bcl-2 and analyzing the same sample, or another biological sample from the same subject, for other markers that are specific indicators of prostate cancer and/or bladder cancer. The one or more additional markers may be detected before, during, and/or after detection of Bcl-2 is carried out. Examples of markers include PSA (prostate specific antigen). The methods, devices, and kits described herein may be modified by including agents to detect the additional markers, or nucleic acids encoding the markers.

Prostate cancer biomarkers that may be used in conjunction with the invention include, but are not limited to: free prostate specific antigen (PSAF), prostate specific antigen-alpha(1) (PSA), prostate specific antigen-alpha(1) antichymotrypsin complex (PSAC), prostatic acid phosphatase (PAP), beta 2 microglobulin (B2M), and alpha 2 microglobulin (A2M).

Bladder cancer biomarkers that may be used in conjunction with the invention include, but are not limited to, chorionic gonadaotropin beta (hCGb).

The selection of biological sample (such as blood or urine) in which the aforementioned cancer markers are diagnostic and/or prognostic can be readily determined by those skilled in the art. As indicated above, the present invention provides a method for monitoring, diagnosing, or for the prognosis of cancer, such as prostate cancer and bladder cancer, in a subject by detecting Bcl-2 in a biological sample from the subject. In an embodiment, the method comprises contacting the sample with an antibody specific for Bcl-2 which is directly or indirectly labeled with a detectable substance, and detecting the detectable substance.

The methods of the invention may be used for the detection of either an over- or an under-abundance of Bcl-2 relative to a non-disorder state or the presence of a modified (e.g., less than full length) Bcl-2 which correlates with a disorder state (e.g., prostate cancer, bladder cancer), or a progression toward a disorder state. The methods described herein may be used to evaluate the probability of the presence of malignant or pre-malignant cells. Such methods can be used to detect malignant tumors, quantitate their growth, and assist in the diagnosis and prognosis of cancer. The methods can be used to detect the presence of cancer metastasis, as well as confirm the absence or removal of all malignant tumor tissue following surgery, cancer chemotherapy, and/or radiation therapy. They can further be used to monitor cancer chemotherapy and malignant tumor reappearance.

The methods of the invention are particularly useful in the diagnosis of early stage prostate cancer (e.g., when the subject is asymptomatic) and for the prognosis of prostate cancer or bladder cancer disease progression and mortality. As illustrated herein, increased levels of Bcl-2 detected in a sample (e.g., urine, serum, plasma, whole blood, ascites) compared to a standard (e.g., levels for normal or benign disorders) are indicative of advanced disease stage, serous histological type, suboptimal debulking, large residual tumor, and/or increased risk of disease progression and mortality.

The terms “sample”, “biological sample”, and the like refer to a type of material known to or suspected of expressing or containing Bcl-2, such as urine. The test sample can be used directly as obtained from the source or following a pretreatment to modify the character of the sample. The sample can be derived from any biological source, such as tissues or extracts, including cells (e.g., tumor cells) and physiological fluids, such as, for example, whole blood, plasma, serum, peritoneal fluid, ascites, and the like.

The sample can be obtained from animals, preferably mammals, most preferably humans. The sample can be pretreated by any method and/or can be prepared in any convenient medium that does not interfere with the assay. The sample can be treated prior to use, such as preparing plasma from blood, diluting viscous fluids, applying one or more protease inhibitors to samples such as urine (e.g., 4-(2 aminoethyl)-benzene sulfonyl fluoride, EDTA, leupeptin, and/or pepstatin), and the like. Sample treatment can involve filtration, distillation, extraction, concentration, inactivation of interfering components, the addition of reagents, and the like.

The presence of bcl-2 may be detected in a variety of biological samples, including tissues or extracts thereof. Preferably, Bcl-2 is detected in human urine.

In embodiments of the invention, the method described herein is adapted for diagnosing and monitoring prostate cancer and/or bladder cancer by quantitating Bcl-2 in biological samples from a subject. Preferably, the amount of Bcl-2 quantitated in a sample from a subject being tested is compared to levels quantitated for another sample or an earlier sample from the subject, or levels quantitated for a control sample. Levels for control samples from healthy subjects may be established by prospective and/or retrospective statistical studies. Healthy subjects who have no clinically evident disease or abnormalities may be selected for statistical studies. Diagnosis may be made by a finding of statistically different levels of Bcl-2 compared to a control sample or previous levels quantitated for the same subject.

The term “Bcl-2” refers to B-cell lymphoma protein 2 (also known as B-cell CLL/lymphoma 2), an integral outer mitochondrial protein that blocks the apoptotic death of some cells such as lymphocytes (Cleary M. L. et al., Cell, 1986, 47(1):19-28; Tsujimoto Y. and Croce C. M., Proc. Natl. Acad. Sci. USA, 1986, 83:5214-5218, which are incorporated herein by reference in their entirety). The term “Bcl-2” includes nucleic acid sequences (e.g., GenBank Accession No. M14745; SEQ ID NO:1) encoding the Bcl-2 gene product (polypeptide), as well as the Bcl-2 polypeptide (e.g., GenBank Accession No. AAA35591; SEQ ID NO:2). In a preferred embodiment, the present invention relates to the use of human Bcl-2 as a biomarker for the diagnosis and/or prognosis of prostate cancer and/or bladder cancer. In one embodiment, the present invention encompasses the use of all homologs, naturally occurring allelic variants, isoforms and precursors of human Bcl-2 of GenBank Accession Nos. M14745 and AAA35591. In general, naturally occurring allelic variants of human Bcl-2 will share significant sequence homology (70-90%) to the sequences shown in GenBank Accession Nos. M14745 and AAA35591. Allelic variants may contain conservative amino acid substitutions from the Bcl-2 sequence or will contain a substitution of an amino acid from a corresponding position in a Bcl-2 homologue. Two transcript variants, alpha and beta, produced by alternative splicing, differ in their C-terminal ends. The alpha variant (GenBank Accession No. NP_{—}000624 (SEQ ID NO:4); and GenBank Accession No. NM_{—}000633 (SEQ ID NO:3)) represents the longer transcript and encodes the longer isoform (alpha), and beta being the shorter (GenBank Accession No. NM_{—}000648 (SEQ ID NO:6); GenBank Accession No. NP_{—}000657 (SEQ ID NO:5). The beta variant differs in the 3′ UTR and coding region compared to the alpha variant, as well as the C-terminal end. In a particular embodiment, the methods, devices, and kits of the invention are specific for Bcl-2 (e.g., SEQ ID NOs: 1, 2, 3, 4, 5, and/or 6), but not nucleic acid molecules or polypeptides known in the art as “Bcl-2-like” molecules (e.g., employing binding agents specific for (e.g., immunoreactive with) Bcl-2, but not reactive with Bcl-2 like molecules), such as those described in Ruben et al., U.S. Patent Application Publication 2002/0106731 A1, published Aug. 8, 2002, which is incorporated herein by reference in its entirety.

The terms “subject” and “patient” are used interchangeably herein to refer to a warm-blooded animal, such as a mammal, which may be afflicted with cancer. Agents that are capable of detecting Bcl-2 in the biological samples of subjects are those that interact or bind with the Bcl-2 polypeptide or the nucleic acid molecule encoding Bcl-2. Examples of such agents (also referred to herein as binding agents) include, but are not limited to, Bcl-2 antibodies or fragments thereof that bind Bcl-2, Bcl-2 binding partners, and nucleic acid molecules that hybridize to the nucleic acid molecules encoding Bcl-2 polypeptides. Preferably, the binding agent is labeled with a detectable substance (e.g., a detectable moiety). The binding agent may itself function as a label.

### Bcl-2 Antibodies

Antibodies specific for Bcl-2 that are used in the methods of the invention may be obtained from scientific or commercial sources. Alternatively, isolated native Bcl-2 or recombinant Bcl-2 may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladne et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Preferably, antibodies used in the methods of the invention are reactive against Bcl-2 if they bind with a Kₐ of greater than or equal to 10⁷ M. In a sandwich immunoassay of the invention, mouse polyclonal antibodies and rabbit polyclonal antibodies are utilized.

In order to produce monoclonal antibodies, a host mammal is inoculated with a Bcl-2 protein or peptide and then boosted. Spleens are collected from inoculated mammals a few days after the final boost. Cell suspensions from the spleens are fused with a tumor cell in accordance with the general method described by Kohler and Milstein (Nature, 1975, 256:495-497). In order to be useful, a peptide fragment must contain sufficient amino acid residues to define the epitope of the Bcl-2 molecule being detected.

If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

Purification of the antibodies or fragments can be accomplished by a variety of methods known to those of skill including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press). It is preferable to use purified antibodies or purified fragments of the antibodies having at least a portion of a Bcl-2 binding region, including such as Fv, F(ab′)₂, Fab fragments (Harlow and Lane, 1988, Antibody Cold Spring Harbor) for the detection of Bcl-2 in the fluids of cancer patients or those at risk, preferably in the urine or blood of prostate cancer and/or or bladder cancer patients.

For use in detection and/or monitoring of cancer, the purified antibodies can be covalently attached, either directly or via linker, to a compound which serves as a reporter group to permit detection of the presence of Bcl-2. A variety of different types of substances can serve as the reporter group, including but not limited to enzymes, dyes, radioactive metal and non-metal isotopes, fluorogenic compounds, fluorescent compounds, etc. Methods for preparation of antibody conjugates of the antibodies (or fragments thereof) of the invention useful for detection, monitoring are described in U.S. Pat. Nos. 4,671,958; 4,741,900 and 4,867,973.

In one aspect of the invention, preferred binding epitopes may be identified from a known Bcl-2 gene sequence and its encoded amino acid sequence and used to generate Bcl-2 antibodies with high binding affinity. Also, identification of binding epitopes on Bcl-2 can be used in the design and construction of preferred antibodies. For example, a DNA encoding a preferred epitope on Bcl-2 may be recombinantly expressed and used to select an antibody which binds selectively to that epitope. The selected antibodies then are exposed to the sample under conditions sufficient to allow specific binding of the antibody to the specific binding epitope on Bcl-2 and the amount of complex formed then detected. Specific antibody methodologies are well understood and described in the literature. A more detailed description of their preparation can be found, for example, in Practical Immunology, Butt, W. R., ed., Marcel Dekker, New York, 1984.

The present invention also contemplates the detection of Bcl-2 antibodies.

### Protein Binding Assays

Antibodies specifically reactive with Bcl-2, or derivatives, such as enzyme conjugates or labeled derivatives, may be used to detect Bcl-2 in various biological samples, for example they may be used in any known immunoassays which rely on the binding interaction between an antigenic determinant of a protein and the antibodies. Examples of such assays are radioimmunoassays, enzyme immunoassay (e.g., ELISA), immunofluorescence, immnunoprecipitation, latex agglutination, hemagglutination, and histochemical tests.

An antibody specific for Bcl-2 can be labeled with a detectable substance and localized in biological samples based upon the presence of the detectable substance. Examples of detectable substances include, but are not limited to, the following radioisotopes (e.g. ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkalline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against Bcl-2. By way of example, if the antibody having specificity against Bcl-2 is a rabbit IgG antibody, the second antibody may be goat anti-rabbit gamma-globulin labeled with a detectable substance as described herein.

Methods for conjugating or labeling the antibodies discussed above may be readily accomplished by one of ordinary skill in the art. (See, for example, Imman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988, regarding methods for conjugating or labeling the antibodies with an enzyme or ligand binding partner).

Time-resolved fluorometry may be used to detect a signal. For example, the method described in Christopoulos T. K. and Diamandis E. P., Anal. Chem., 1992:64:342-346 may be used with a conventional time-resolved fluorometer.

Therefore, in accordance with an embodiment of the invention, a method is provided wherein a Bcl-2 antibody is labeled with an enzyme, a substrate for the enzyme is added wherein the substrate is selected so that the substrate, or a reaction product of the enzyme and substrate, forms fluorescent complexes with a lanthanide metal. A lanthanide metal is added and Bcl-2 is quantitated in the sample by measuring fluorescence of the fluorescent complexes. The antibodies specific for Bcl-2 may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkalline phosphatase and beta-galactosidase. Preferably, the enzyme is akline phosphatase. The Bcl-2 antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the method, antibody bound to Bcl-2 in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkalline phosphatase, the substrate employed in the method may be 4-methylumbeliferyl phosphate, or 5-fluorpsalicyl phosphate. The fluorescence intensity of the complexes is typically measured using a time-resolved fluorometer, e.g., a CyberFluor 615 Immoanalyzer (Nordion International, Kanata Ontario).

The sample, antibody specific for Bcl-2, or Bcl-2, may be immobilized on a carrier. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling.

In accordance with an embodiment, the present invention provides a mode for determining Bcl-2 in an appropriate sample such as urine by measuring Bcl-2 by immunoassay. It will be evident to a skilled artisan that a variety of immunoassay methods can be used to measure Bcl-2. In general, a Bcl-2 immunoassay method may be competitive or noncompetitive. Competitive methods typically employ an immobilized or immobilizable antibody to Bcl-2 (anti-Bcl-2) and a labeled form of Bcl-2. Sample Bcl-2 and labeled Bcl-2 compete for binding to anti-Bcl-2. After separation of the resulting labeled Bcl-2 that has become bound to anti-Bcl-2 (bound fraction) from that which has remained unbound (unbound fraction), the amount of the label in either bound or unbound fraction is measured and may be correlated with the amount of Bcl-2 in the biological sample in any conventional manner, e.g., by comparison to a standard curve.

Preferably, a noncompetitive method is used for the determination of Bcl-2, with the most common method being the “sandwich” method. In this assay, two anti-Bcl-2 antibodies are employed. One of the anti-Bcl-2 antibodies is directly or indirectly labeled (also referred to as the “detection antibody”) and the other is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises Bcl-2 bound by (“sandwiched” between) the capture and detection antibodies.

In a typical two-site immunometric assay for Bcl-2, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase such as a magnetic particle, a latex particle, a microtiter multi-well plate, a bead, a cuvette, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In an embodiment, the capture antibody can be immobilized using a species specific antibody for the capture antibody that is bound to the solid phase.

A particular sandwich immunoassay method of the invention employs two antibodies reactive against Bcl-2, a second antibody having specificity against an antibody reactive against Bcl-2 labeled with an enzymatic label, and a fluorogenic substrate for the enzyme. In an embodiment, the enzyme is alkalline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

The protein detection methods, devices, and kits of the invention can utilize nanowire sensor technology (Zhen et al., Nature Biotechnology, 2005, 23(10):1294-1301; Lieber et al., Anal. Chem., 2006, 78(13):4260-4269, which are incorporated herein by reference) or microcantilever technology (Lee et al., Biosens. Bioelectron, 2005, 20(10):2157-2162; Wee et al., Biosens. Bioelectron., 2005, 20(10):1932-1938; Campbell and Mutharasan, Biosens. Bioelectron., 2005, 21(3):462-473; Campbell and Mutharasan, Biosens. Bioelectron., 2005, 21(4):597-607; Hwang et al., Lab Chip, 2004, 4(6):547-552; Mukhopadhyay et al., Nano. Lett., 2005, 5(12):2835-2388, which are incorporated herein by reference) for detection of Bcl-2 in samples. In addition, Huang et al. describe a prostate specific antigen immunoassay on a commercially available surface plasmon resonance biosensor (Biosens. Bioelectron., 2005, 21(3):483-490, which is incorporated herein by reference) which may be adapted for detection of Bcl-2. High-sensitivity miniaturized immunoassays may also be utilized for detection of Bcl-2 (Cesaro-Tadic et al., Lab Chip, 2004, 4(6):563-569; Zimmerman et al., Biomed. Microdevices, 2005, 7(2):99-110, which are incorporated herein by reference).

### Nucleic Acids

Nucleic acids including naturally occurring nucleic acids, oligonucleotides, antisense oligonucleotides, and synthetic oligonucleotides that hybridize to the nucleic acid encoding Bcl-2, are useful as agents for the diagnosis and/or prognosis of prostate cancer and/or bladder cancer. The present invention contemplates the use of nucleic acid sequences corresponding to the coding sequence of Bcl-2 and to the complementary sequence thereof, as well as sequences complementary to the Bcl-2 transcript sequences occurring further upstream or downstream from the coding sequence (e.g., sequences contained in, or extending into, the 5′ and 3′ untranslated regions) for use as agents for detecting the expression of Bcl-2 in biological samples of cancer patients, or those at risk of cancer, preferably in the urine of prostate cancer and/or or bladder cancer patients or those at risk of cancer and/or or bladder prostate cancer.

The preferred oligonucleotides for detecting the presence of Bcl-2 in biological samples are those that are complementary to at least part of the cDNA sequence encoding Bcl-2. These complementary sequences are also known in the art as “antisense” sequences. These oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosohodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding Bcl-2 may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding Bcl-2, oligonucleotides typically within the range of 8-100 nucleotides are preferred. Most preferable oligonucleotides for use in detecting Bcl-2 in urine samples are those within the range of 15-50 nucleotides.

The oligonucleotide selected for hybridizing to the Bcl-2 nucleic acid molecule, whether synthesized chemically or by recombinant DNA technology, is then isolated and purified using standard techniques and then preferably labeled (e.g., with ³⁵S or ³²P) using standard labeling protocols.

The present invention also contemplates the use of oligonucleotide pairs in polymerize chain reactions (PCR) to detect the expression of Bcl-2 in biological samples. The oligonucleotide pairs include a forward Bcl-2 primer and a reverse Bcl-2 primer.

The presence of Bcl-2 in a sample from a patient may be determined by nucleic acid hybridization, such as but not limited to Northern blot analysis, dot blotting, Southern blot analysis, fluorescence in situ hybridization (FISH), and PCR. Chromatography, preferably HPLC, and other known assays may also be used to determine messenger RNA levels of Bel-2 in a sample.

The Bcl-2 encoding nucleic acid molecules conceivably may be found in the biological fluids inside a Bcl-positive cancer cell that is being shed or released in the fluid under investigation.

In one aspect, the present invention contemplates the use of nucleic acids as agents for detecting Bcl-2 in biological samples of patients, wherein the nucleic acids are labeled. The nucleic agents may be labeled with a radioactive label, a fluorescent label, an enzyme, a chemiluminescent tag, a colorimetric tag or other labels or tags that are discussed above or that are known in the art.

In another aspect, the present invention contemplates the use of Northern blot analysis to detect the presence of Bcl-2 mRNA in a sample of bodily fluid. The first step of the analysis involves separating a sample containing Bcl-2 nucleic acid by gel electrophoresis. The dispersed nucleic acids are then transferred to a nitrocellulose filter or another filter. Subsequently, the labeled oligonucleotide is exposed to the filter under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe based microarrays. Measuring the radioactivity of hybridized fragments, using standard procedures known in the art quantitates the amount of Bcl-2 nucleic acid present in the biological fluid of a patient.

Dot blotting involves applying samples containing the nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more specific nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan to detect the presence of desired sequence (U.S. Pat. No. 4,683,195) routinely uses polymerase chain reaction.

A specific example of PCR that is routinely performed by the skilled artisan to detect desired sequences is reverse transcript PCR (RT-PCR; Saiki et al., Science, 1985, 230:1350; Scharf et al., Science, 1986, 233:1076). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

In a preferred embodiment, the methods of detecting Bcl-2 nucleic acid in biological fluids of cancer patients or those at risk thereof, preferably urine of prostate cancer and/or or bladder cancer patients or those at risk thereof, include Northern blot analysis, dot blotting, Southern blot analysis, FISH, and PCR.

### Devices

The methods of the invention can be carried out on a solid support. The solid supports used may be those which are conventional for the purpose of assaying an analyte in a biological sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. Bcl-2 can be detected in a sample in vivo or in vitro (ex vivo). When, according to an embodiment of the invention, the amount of Bcl-2 in a sample is to be determined without removing the sample from the body (i.e., in vivo), the support should be one which is harmless to the subject and may be in any form convenient for insertion into an appropriate part of the body. For example, the support may be a probe made of polytetrafluoroethylene, polystyrene or other rigid non-harmful plastic material and having a size and shape to enable it to be introduced into a subject. The selection of an appropriate inert support is within the competence of those skilled in the art, as are its dimensions for the intended purpose.

A contacting step in the assay (method) of the invention can involve contacting, combining, or mixing the biological sample and the solid support, such as a reaction vessel, microvessel, tube, microtube, well, multi-well plate, or other solid support. In an embodiment of the invention, the solid support to be contacted with the biological sample (e.g., urine) has an absorbent pad or membrane for lateral flow of the liquid medium to be assayed, such as those available from Millipore Corp. (Bedford, Mass.), including but not limited to Hi-Flow Plus™ membranes and membrane cards, and SureWick™ pad materials.

The diagnostic device useful in carrying out the methods of the invention can be constructed in any form adapted for the intended use. Thus, in one embodiment, the device of the invention can be constructed as a disposable or reusable test strip or stick to be contacted with a biological sample such as urine or blood for which Bcl-2 level is to be determined. In another embodiment, the device can be constructed using art recognized micro-scale manufacturing techniques to produce needle-like embodiments capable of being implanted or injected into an anatomical site, such as the peritoneal cavity, for indwelling diagnostic applications. In other embodiments, devices intended for repeated laboratory use can be constructed in the form of an elongated probe.

In preferred embodiments, the devices of the invention comprise a solid support (such as a strip or dipstick), with a surface that functions as a lateral flow matrix defining a flow path for a biological sample such as urine, whole blood, serum, plasma, peritoneal fluid, or ascites.

Immunochromatographic assays, also known as lateral flow test strips or simply strip tests, for detecting various analytes of interest, have been known for some time, and may be used for detection of Bcl-2. The benefits of lateral flow tests include a user-friendly format, rapid results, long-term stability over a wide range of climates, and relatively low cost to manufacture. These features make lateral flow tests ideal for applications involving home testing, rapid point of care testing, and testing in the field for various analytes. The principle behind the test is straightforward. Essentially, any ligand that can be bound to a visually detectable solid support, such as dyed microspheres, can be tested for, qualitatively, and in many cases even semi-quantitatively. For example, a one-step lateral flow immunostrip for the detection of free and total prostate specific antigen in serum is described in Fernandez-Sanchez et al. (J. Immuno. Methods, 2005, 307(1-2):1-12, which is incorporated herein by reference) and may be adapted for detection of Bcl-2 in a biological sample such as blood or urine.

Some of the more common immunochromatographic assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbiol, 2001, 39:3495-3498). Human immunodeficiency virus (HIV) has been detected rapidly in pooled blood using a similar assay (Soroka, S. D. et al. J Clin Virol, 2003, 27:90-96). A nitrocellulose membrane card has also been used to diagnose schistosomiasis by detecting the movement and binding of nanoparticles of carbon (van Dam, G. J. et al. J Clin Microbiol, 2004, 42:5458-5461).

The two common approaches to the immunochromatographic assay are the non-competitive (or direct) and competitive (or competitive inhibition) reaction schemes (TechNote #303, Rev. #001, 1999, Bangs Laboratories, Inc., Fishers, Ind.). The direct (double antibody sandwich) format is typically used when testing for larger analytes with multiple antigenic sites such as luteinizing hormone (LH), human chorionic gonadotropin (hCG), and HIV. In this instance, less than an excess of sample analyte is desired, so that some of the microspheres will not be captured at the capture line, and will continue to flow toward the second line of immobilized antibodies, the control zone. This control line uses species-specific anti-immunoglobulin antibodies, specific for the conjugate antibodies on the microspheres. Free antigen, if present, is introduced onto the device by adding sample (urine, serum, etc.) onto a sample addition pad. Free antigen then binds to antibody-microsphere complexes. Antibody 1, specific for epitope 1 of sample antigen, is coupled to dye microspheres and dried onto the device. When sample is added, microsphere-antibody complex is rehydrated and carried to a capture zone and control lines by liquid. Antibody 2, specific for a second antigenic site (epitope 2) of sample antigen, is dried onto a membrane at the capture line. Antibody 3, a species-specific, anti-immunoglobulin antibody that will react with antibody 1, is dried onto the membrane at the control line. If antigen is present in the sample (i.e., a positive test), it will bind by its two antigenic sites, to both antibody 1 (conjugated to microspheres) and antibody 2 (dried onto membrane at the capture line). Antibody 1-coated microspheres are bound by antibody 3 at the control line, whether antigen is present or not. If antigen is not present in the sample (a negative test), microspheres pass the capture line without being trapped, but are caught by the control line.

The competitive reaction scheme is typically used when testing for small molecules with single antigenic determinants, which cannot bond to two antibodies simultaneously. As with double antibody sandwich assay, free antigen, if present is introduced onto the device by adding sample onto a sample pad. Free antigen present in the sample binds to an antibody-microsphere complex. Antibody 1 is specific for sample antigen and couple to dyed microspheres. An antigen-carrier molecule (typically BSA) conjugate is dried onto a membrane at the capture line. Antibody 2 (Ab2) is dried onto the membrane at the control line, and is a species-specific anti-immunoglobulin that will capture the reagent particles and confirm that the test is complete. If antigen is present in the sample (a positive test), antibody on microspheres (Ab1) is already saturated with antigen from sample and, therefore, antigen conjugate bound at the capture line does not bind to it. Any microspheres not caught by the antigen carrier molecule can be caught by Ab2 on the control line. If antigen is not present in the sample (a negative test), antibody-coated dyed microspheres are allowed to be captured by antigen conjugate bound at the capture line.

Normally, the membranes used to hold the antibodies in place on these devices are made of primary hydrophobic materials, such as nitrocellulose. Both the microspheres used as the solid phase supports and the conjugate antibodies are hydrophobic, and their interaction with the membrane allows them to be effectively dried onto the membrane.

Samples and/or Bcl-2-specific binding agents may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or -1536 well, plates (or trays). Optionally, Bcl-2-specific binding agents may be immobilized on the solid support.

Detection of Bcl-2 and cancer biomarkers, and other assays that are to be carried out on samples, can be carried out simultaneously or sequentially with the detection of other target molecules, and may be carried out in an automated fashion, in a high-throughput format.

The Bcl-2-specific binding agents can be deposited but “free” (non-immobilized) in the conjugate zone, and be immobilized in the capture zone of a solid support. The Bcl-2-specific binding agents may be immobilized by non-specific adsorption onto the support or by covalent bonding to the support, for example. Techniques for immobilizing binding agents on supports are known in the art and are described for example in U.S. Pat. Nos. 4,399,217, 4,381,291, 4,357,311, 4,343,312 and 4,260,678, which are incorporated herein by reference. Such techniques can be used to immobilize the binding agents in the invention. When the solid support is polytetrafluoroethylene, it is possible to couple hormone antibodies onto the support by activating the support using sodium and ammonia to aminate it and covalently bonding the antibody to the activated support by means of a carbodiimide reaction (yon Klitzing, Schultek, Strasburger, Fricke and Wood in “Radioimmunoassay and Related Procedures in Medicine 1982”, International Atomic Energy Agency, Vienna (1982), pages 57-62.).

The diagnostic device of the invention can utilize lateral flow strip (LFS) technology, which has been applied to a number of other rapid strip assay systems, such as over-the-counter early pregnancy test strips based on antibodies to human chorionic gonadotropin (hCG). As with many other diagnostic devices, the device utilizes a binding agent to bind the target molecule (Bcl-2). The device has an application zone for receiving a biological sample such as blood or urine, a labeling zone containing label which binds to Bcl-2 in the sample, and a detection zone where Bcl-2 label is retained.

Binding agent retained in the detection zone gives a signal, and the signal differs depending on whether Bcl-2 levels in the biological sample are lower than, equal to, or greater than a given threshold concentration.

For example, in the use of urinary Bcl-2 for the detection of prostate cancer, the threshold concentration may be 12 ng/ml or higher than 12 ng/ml (including but not limited to, 13, 14, 15, 16, 17, 18, 19, 20, 21, 22, 23, 24, 25, 26, 27, 28, 29, 30 ng/ml, or higher). In one embodiment, for the detection of prostate cancer, the threshold urinary Bcl-2 concentration is 12 ng/ml. In one embodiment, for the detection of prostate cancer, the threshold urinary Bcl-2 concentration is 15 ng/ml.

For example, in the use of urinary Bcl-2 for the detection of bladder cancer, the threshold concentration may be 12 ng/ml or higher than 12 ng/ml (including but not limited to, 13, 14, 15, 16, 17, 18, 19, 20, 21, 22, 23, 24, 25, 26, 27, 28, 29, 30 ng/ml, or higher). In one embodiment, for the detection of bladder cancer, the threshold urinary Bcl-2 concentration is 12 ng/ml. In one embodiment, for the detection of bladder cancer, the threshold urinary Bcl-2 concentration is 16 ng/ml.

A sample from a subject having a Bcl-2 level equal to or greater than the given reference Bcl-2 concentration can be referred to as a “threshold level”, “threshold amount”, or “threshold sample”. The application zone in the device is suitable for receiving the biological sample to be assayed. It is typically formed from absorbent material such as blotting paper. The labeling zone contains binding agent that binds to any Bcl-2 in the sample. In one embodiment, the binding agent is an antibody (e.g., monoclonal antibody, polyclonal antibody, antibody fragment). For ease of detection, the binding agent is preferably in association with a label that provides a signal that is visible to the naked eye, e.g., it is tagged with a fluorescent tag or a colored tag such as conjugated colloidal gold, which is visible as a pink color.

The detection zone retains Bcl-2 to which the binding agent has bound. This will typically be achieved using an immobilized binding agent such as an immobilized antibody. Where the binding agent in the labeling zone and the detection zone are both antibodies, they will typically recognize different epitopes on the target molecule (Bcl-2 protein). This allows the formation of a “sandwich” comprising antibody-Bcl-2-antibody.

The detection zone is downstream of the application zone, with the labeling zone typically located between the two. A sample will thus migrate from the application zone into the labeling zone, where any in the sample binds to the label. Bcl-2-binding agent complexes continue to migrate into the detection zone together with excess binding agent. When the Bcl-2-binding agent complex encounters the capture reagent, the complex is retained whilst the sample and excess binding agent continue to migrate. As Bcl-2 levels in the sample increase, the amount of binding agent (in the form of Bcl-2-binding agent complex) retained in the detection zone increases proportionally.

In preferred embodiments, the device of the invention has the ability to distinguish between samples according to the threshold concentration. This can be achieved in various ways.

One type of device includes a reference zone that includes a signal of fixed intensity against which the amount of binding agent retained in the detection zone can be compared—when the signal in the detection zone equals the signal in the reference zone, the sample is a threshold sample; when the signal in the detection zone is less intense than the reference zone, the sample contains less Bcl-2 than a threshold sample; when the signal in the detection zone is more intense than the reference zone, the sample contains more Bcl-2 than a threshold sample.

A suitable reference zone can be prepared and calibrated without difficulty. For this type of device, the binding agent will generally be present in excess to Bcl-2 in the sample, and the reference zone may be upstream or, preferably, downstream of the detection zone. The signal in the reference zone will be of the same type as the signal in the detection zone, i.e., they will typically both be visible to the naked eye, e.g., they will use the same tag. A preferred reference zone in a device of this type comprises immobilized protein (e.g., bovine serum albumin) which is tagged with colloidal gold.

In another device of the invention, the reference zone is downstream of the detection zone and includes a reagent which captures binding agent (e.g., an immobilized anti-binding agent antibody). Binding agent that flows through the device is not present in excess, but is at a concentration such that 50% of it is bound by a sample having Bcl-2 at the threshold concentration. In a threshold sample, therefore, 50% of the binding agent will be retained in the detection zone and 50% in the reference zone. If the Bcl-2 level in the sample is greater than in a threshold sample, less than 50% of the binding agent will reach the reference zone and the detection zone will give a more intense signal than the reference zone; conversely, if the Bcl-2 level in the sample is less than in a threshold sample, less than 50% of the binding agent will be retained in the detection zone and the reference zone will give a more intense signal than the detection zone.

In another device of the invention which operates according to similar principles, the reference zone is downstream of the detection zone and includes a limiting amount of a reagent which captures binding agent (e.g., an immobilized anti-binding agent antibody). The reagent is present at a level such that it retains the same amount of label which would bind to the detection zone for a threshold sample, with excess label continuing to migrate beyond the reference zone.

In these three types of device, therefore, a comparison between the detection zone and the reference zone is used to compare the sample with the threshold concentration. The detection: reference binding ratio can preferably be determined by eye. Close juxtaposition of the detection and reference zones is preferred in order to facilitate visual comparison of the signal intensities in the two zones.

In a fourth type of device, no reference zone is needed, but the detection zone is configured such that it gives an essentially on/off response, e.g., no signal is given below the threshold concentration but, at or above the threshold, signal is given.

In a fifth type of device, no reference zone is needed, but an external reference is used which corresponds to the threshold concentration. This can take various forms, e.g., a printed card against which the signal in the detection zone can be compared, or a machine reader which compares an absolute value measured in the detection zone (e.g., a calorimetric signal) against a reference value stored in the machine.

In some embodiments of the invention, the device includes a control zone downstream of the detection zone. This will generally be used to capture excess binding agent that passes through the detection and/or reference zones (e.g., using immobilized anti-binding agent antibody). When binding agent is retained at the control zone, this confirms that mobilization of the binding agent and migration through the device have both occurred. It will be appreciated that this function may be achieved by the reference zone.

In a preferred embodiment, the detection, reference and control zones are preferably formed on a nitrocellulose support.

Migration from the application zone to the detection zone will generally be assisted by a wick downstream of the detection zone to aid capillary movement. This wick is typically formed from absorbent material such as blotting or chromatography paper.

The device of the invention can be produced simply and cheaply, conveniently in the form of a dipstick. Furthermore, it can be used very easily, for instance by the home user. The invention thus provides a device which can be used at home as a screen for cancer, such as prostate cancer and/or or bladder cancer.

### Kits for Diagnosing or Monitoring Cancer

In one aspect, the present invention includes kits comprising the required elements for diagnosing or monitoring cancer. Preferably, the kits comprise a container for collecting biological fluid from a patient and an agent for detecting the presence of Bcl-2 or its encoding nucleic acid in the fluid. The components of the kits can be packaged either in aqueous medium or in lyophilized form.

The methods of the invention can be carried out using a diagnostic kit for qualitatively or quantitatively detecting Bcl-2 in a sample such as blood or urine. By way of example, the kit can contain binding agents (e.g., antibodies) specific for Bcl-2, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit. In one embodiment, the kit includes one or protease inhibitors (e.g., a protease inhibitor cocktail) to be applied to the biological sample to be assayed (such as blood or urine).

Kits for diagnosing or monitoring cancer containing one or more agents that detect the Bcl-2 protein, such as but not limited to Bcl-2 antibodies, fragments thereof, or Bcl-2 binding partners, can be prepared. The agent(s) can be packaged with a container for collecting the biological fluid from a patient. When the antibodies or binding partner are used in the kits in the form of conjugates in which a label is attached, such as a radioactive metal ion or a moiety, the components of such conjugates can be supplied either in fully conjugated form, in the form of intermediates or as separate moieties to be conjugated by the user of the kit.

Kits containing one or more agents that detect Bcl-2 nucleic acid, such as but not limited to the full length Bcl-2 nucleic acid, Bcl-2 oligonucleotides, and pairs of Bcl-2 primers can also be prepared. The agent(s) can be packaged with a container for collecting biological samples from a patient. The nucleic acid can be in the labeled form or to be labeled form.

Other components of the kit may include but are not limited to, means for collecting biological samples, means for labeling the detecting agent (binding agent), membranes for immobilizing the Bcl-2 protein or Bcl-2 nucleic acid in the biological sample, means for applying the biological sample to a membrane, means for binding the agent to Bcl-2 in the biological sample of a subject, a second antibody, a means for isolating total RNA from a biological fluid of a subject, means for performing gel electrophoresis, means for generating cDNA from isolated total RNA, means for performing hybridization assays, and means for performing PCR, etc.

As used herein, the term “ELISA” includes an enzyme-linked immunosorbent assay that employs an antibody or antigen bound to a solid phase and an enzyme-antigen or enzyme-antibody conjugate to detect and quantify the amount of an antigen (e.g., Bcl-2) or antibody present in a sample. A description of the ELISA technique is found in Chapter 22 of the 4^{th} Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other sample of interest such as urine can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

### DEFINITIONS

Cancer is a malignant neoplasm that involves unregulated cell growth. In cancer, cells divide and grow uncontrollably, forming malignant tumors, and invade nearby parts of the body. Not all tumors are cancerous. Benign tumors do not grow uncontrollably, do not invade neighboring tissues, and do not spread throughout the body.

As used herein, the term “tumor” refers to all neoplastic cell growth, and includes, benign, pre-malignant, and malignant cells and tissues. A primary tumor mass refers to a tumor that is at the original site where it first arose. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), ultrasound, CT, and MRI, or by needle aspirations. The use of these latter techniques is more common in early detection. Molecular and phenotypic analysis of cancer cells within a tissue will usually confirm if the cancer is endogenous to the tissue or if the lesion is due to metastasis from another site.

A “sample” (biological sample) can be any composition of matter of interest from a human or non-human subject, in any physical state (e.g., solid, liquid, semi-solid, vapor) and of any complexity. The sample can be any composition reasonably suspecting of containing Bcl-2 that can be analyzed by the methods, devices, and kits of the invention. Preferably, the sample is a fluid (biological fluid). Samples can include human or animal samples. The sample may be contained within a test tube, culture vessel, multi-well plate, or any other container or supporting substrate. The sample can be, for example, a cell culture, human or animal tissue. Fluid homogenates of cellular tissues are biological fluids that may contain Bcl-2 for detection by the invention.

The “complexity” of a sample refers to the relative number of different molecular species that are present in the sample.

The terms “body fluid” and “bodily fluid”, as used herein, refer to a composition obtained from a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions or mixtures containing homogenized solid material, such as feces, tissues, and biopsy samples.

The term “ex vivo,” as used herein, refers to an environment outside of a subject. Accordingly, a sample of bodily fluid collected from a subject is an ex vivo sample of bodily fluid as contemplated by the subject invention. In-dwelling embodiments of the method and device of the invention obtain samples in vivo.

As used herein, the term “conjugate” refers to a compound comprising two or more molecules bound together, optionally through a linking group, to form a single structure. The binding can be made by a direct connection (e.g., a chemical bond) between the molecules or by use of a linking group.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., Hi-Flow™ Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

As used herein, the term “receptor” and “receptor protein” are used herein to indicate a biologically active proteinaceous molecule that specifically binds to (or with) other molecules such as Bcl-2.

As used herein, the term “ligand” refers to a molecule that contains a structural portion that is bound by specific interaction with a particular receptor protein.

As used herein, the term “antibody” refers to immunoglobulin molecules and immunologically active portions (fragments) of immunoglobulin molecules, i.e., molecules that contain an antibody combining site or paratope. The term is inclusive of monoclonal antibodies and polyclonal antibodies.

As used here, the terms “monoclonal antibody” or “monoclonal antibody composition” refer to an antibody molecule that contains only one species of antibody combining site capable of immunoreacting with a particular antigen. A monoclonal antibody composition thus typically displays a single binding affinity for any antigen with which it immunoreacts. A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281. The references cited are hereby incorporated herein by reference.

As used herein, a semi-permeable membrane refers to a bio-compatible material which is impermeable to liquids and capable of allowing the transfer of gases through it. Such gases include, but are not limited to, oxygen, water vapor, and carbon dioxide. Semi-permeable membranes are an example of a material that can be used to form a least a portion of an enclosure defining a flow chamber cavity. The semi-permeable membrane may be capable of excluding microbial contamination (e.g., the pore size is characteristically small enough to exclude the passage of microbes that can contaminate the analyte, such as cells). In a particular aspect, a semi-permeable membrane can have an optical transparency and clarity sufficient for permitting observation of an analyte, such as cells, for color, growth, size, morphology, imaging, and other purposes well known in the art.

As used herein, the term “bind” refers to any physical attachment or close association, which may be permanent or temporary. The binding can result from hydrogen bonding, hydrophobic forces, van der Waals forces, covalent, or ionic bonding, for example.

As used herein, the term “particle” includes insoluble materials of any configuration, including, but not limited to, spherical, thread-like, brush-like, and irregular shapes. Particles can be porous with regular or random channels inside. Particles can be magnetic. Examples of particles include, but are not limited to, silica, cellulose, Sepharose beads, polystyrene (solid, porous, derivatized) beads, controlled-pore glass, gel beads, magnetic beads, sols, biological cells, subcellular particles, microorganisms (protozoans, bacteria, yeast, viruses, and other infectious agents), micelles, liposomes, cyclodextrins, and other insoluble materials.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences.

As used herein, the term “polypeptide” refers to any polymer comprising any number of two or more amino acids, and is used interchangeably herein with the terms “protein”, “gene product”, and “peptide”.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide”, “nucleic acid molecule”, and “nucleotide molecule” are used interchangeably herein and refer to a polymer of nucleotides joined together by a phosphodiester linkage between 5′ and 3′ carbon atoms. Polynucleotides can encode a polypeptide such as Bcl-2 polypeptide (whether expressed or non-expressed), or may be short interfering RNA (siRNA), antisense nucleic acids (antisense oligonucleotides), aptamers, ribozymes (catalytic RNA), or triplex-forming oligonucleotides (i.e., antigene), for example.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog” or “nucleic acid analog”, also referred to herein as an altered nucleotide/nucleic acid or modified nucleotide/nucleic acid refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. For example, locked nucleic acids (LNA) are a class of nucleotide analogs possessing very high affinity and excellent specificity toward complementary DNA and RNA. LNA oligonucleotides have been applied as antisense molecules both in vitro and in vivo (Jepsen J. S. et al., Oligonucleotides, 2004, 14(2):130-146).

As used herein, the term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA).

The terms “comprising”, “consisting of” and “consisting essentially of” are defined according to their standard meaning The terms may be substituted for one another throughout the instant application in order to attach the specific meaning associated with each term.

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “an antibody” includes more than one such antibody. A reference to “a molecule” includes more than one such molecule, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

### EXAMPLE

Following is an example that illustrates materials, methods, procedures, and embodiments for practicing the invention. The example is illustrative and should not be construed as limiting.

### Materials and Methods

### Patient Cohort.

With prior institutional approval, urine samples were collected from normal healthy control volunteers and patients with cancer (N=5 per cancer type) at the H. Lee Moffitt Cancer Center. Specimens were collected prior to initial surgical debulking,

### Sample Preparation.

With patient informed consent, urine samples were collected from patients, anonymized and coded to protect patient identity, and released from the H. Lee Moffitt Cancer Center for this research protocol. All samples were kept in ice. Urine samples were treated with a standard protease inhibitor cocktail (80 μg/ml 4-(2 aminoethyl)-benzene sulfonyl fluoride, 200 μg/ml EDTA, 0.2 μg/ml leupeptin, 0.2 μg/ml pepstatin, Sigma Scientific, St. Louis, Mich.) and centrifuged at 3000×g. Urinary supernates and plasma samples were then aliquoted and stored at −20° C.

### Slot Blot Analysis.

To measure Bcl-2 protein levels in patients' urine, samples were assayed using Slot blots were performed using the Bio-Rad Bio-Dot SF® microfiltration apparatus according to the manufacturer's instructions. Membranes were incubated with anti Bcl-2 antibodies according to standard western blot procedure and visualized with enhanced chemiluminescence. The values are average densitometric values from 5 samples per cancer type. Urinary Bcl-2 was tested using slot blots with a Bcl-2 standard curve.

### Example 1

### Diagnosis of Prostate and Bladder Cancer Based on Elevated Urinary Bcl-2 Level

Urine samples were collected from normal controls (N=21) and patients with cancer (N=5 each cancer type). The latter category consisted of patients diagnosed with sarcoma, lung cancer, brain cancer, cervical cancer, endometrial cancer, colon cancer, breast cancer, melanoma, prostate cancer, ovarian cancer, and bladder cancer.

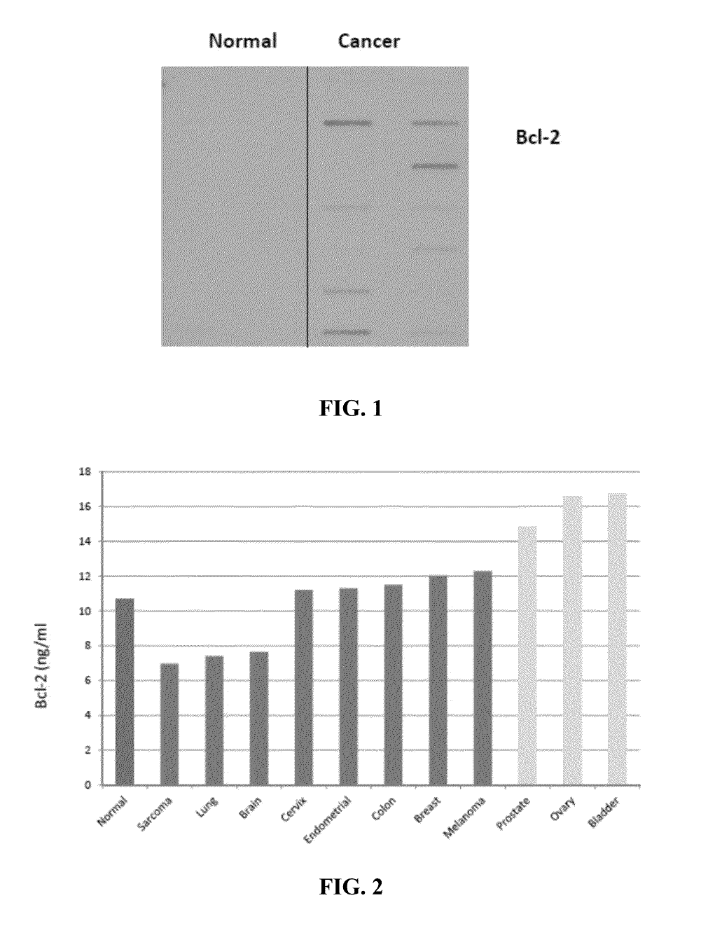

Urinary levels of Bcl-2 of samples from the normal controls and patients with cancer were determined by Slot Blot analyses (FIG. 1 and FIG. 2). The level of urinary Bcl-2 was 12 ng/ml in normal control samples. Urinary Bcl-2 levels were significantly elevated in patients with prostate cancer (15 ng/ml), ovarian cancer (above 16 ng/ml), and bladder cancer (above 16 ng/ml).

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method of diagnosing prostate cancer in a subject, comprising:
determining a Bcl-2 level in a urine sample from a subject;
determining whether the level of Bcl-2 protein in the subject's sample exceeds a predetermined threshold representing the level of urinary Bcl-2 protein in a non-cancerous individual, wherein the subject's Bcl-2 level higher than the pre-determined threshold level of urinary Bcl-2 protein of a non-cancerous individual indicates that the subject has prostate cancer.

2. The method according to claim 1, further comprising: if the subject's Bcl-2 level is higher than the pre-determined threshold level of urinary Bcl-2 protein of a non-cancerous individual, then performing at least one of the following steps:
performing at least one additional diagnostic test or examination for prostate cancer;
informing the subject as having prostate cancer; and/or
prescribing treatment for prostate cancer; and
if the subject's Bcl-2 level is not higher than the pre-determined threshold level of urinary Bcl-2 protein of a non-cancerous individual, then performing at least one of the following steps:
informing the subject as not having prostate cancer; and/or
not prescribing treatment for prostate cancer.

3. The method according to claim 1, wherein the subject's Bcl-2 level is the level of Bcl-2 protein.

4. The method according to claim 3, wherein the subject's Bcl-2 protein level is measured by enzyme-linked immunosorbent assay (ELISA).

5. The method of claim 3, wherein said detecting comprises:
(a) contacting the biological sample with a binding agent that binds Bcl-2 protein to form a complex;
(b) detecting the complex; and
(c) correlating the detected complex to the amount of Bcl-2 protein in the sample.

6. The method of claim 3, wherein the binding agent is immobilized on a support.

7. The method of claim 3, wherein the binding agent is a monoclonal or polyclonal antibody.

8. The method of claim 3, wherein said detecting of (b) further comprises linking or incorporating a label onto the binding agent.

9. The method of claim 3, wherein the pre-determined Bcl-2 threshold level is at least 12 ng/ml.

10. A method of diagnosing bladder cancer in a subject, comprising:
determining a Bcl-2 level in a urine sample from a subject;
determining whether the level of Bcl-2 protein in the subject's sample exceeds a predetermined threshold representing the level of urinary Bcl-2 protein in a non-cancerous individual, wherein the subject's Bcl-2 level higher than the pre-determined threshold level of urinary Bcl-2 protein of a non-cancerous individual indicates that the subject has bladder cancer.

11. The method according to claim 10, further comprising: if the subject's Bcl-2 level is higher than the pre-determined threshold level of urinary Bcl-2 protein of a non-cancerous individual, then performing at least one of the following steps:
performing at least one additional diagnostic test or examination for bladder cancer;
informing the subject as having bladder cancer; and/or
prescribing treatment for bladder cancer; and
if the subject's Bcl-2 level is not higher than the pre-determined threshold level of urinary Bcl-2 protein of a non-cancerous individual, then performing at least one of the following steps:
informing the subject as not having bladder cancer; and/or
not prescribing treatment for bladder cancer.

12. The method according to claim 10, wherein the subject's Bcl-2 level is the level of Bcl-2 protein.

13. The method according to claim 12, wherein the subject's Bcl-2 protein level is measured by enzyme-linked immunosorbent assay (ELISA).

14. The method of claim 12, wherein said detecting comprises:
(a) contacting the biological sample with a binding agent that binds Bcl-2 protein to form a complex;
(b) detecting the complex; and
(c) correlating the detected complex to the amount of Bcl-2 protein in the sample.

15. The method of claim 12, wherein the binding agent is immobilized on a support.

16. The method of claim 12, wherein the binding agent is a monoclonal or polyclonal antibody.

17. The method of claim 12, wherein said detecting of (b) further comprises linking or incorporating a label onto the binding agent.

18. The method of claim 12, wherein the pre-determined Bcl-2 threshold level is at least 12 ng/ml.

19. A device for the rapid detection of Bcl-2 in a sample of urine, comprising an application zone for receiving a urine sample; a labeling zone containing a binding agent that binds to Bcl-2 in the sample; and a detection zone where Bcl-2-bound binding agent is retained to give a signal, wherein the device is configured such that the signal given for a sample from a subject with a Bcl-2 level no greater than a threshold concentration is different from the signal given for a sample from a subject with a Bcl-2 level greater than a threshold concentration, wherein said threshold concentration is at least 12 ng/ml.

20. The device of claim 19, wherein the device has a reference zone which gives a signal which has the same intensity as the signal given in the detection zone for a sample from a subject having a Bcl-2 level equal to the threshold concentration.


SEQUENCE LISTING (6 sequences)

SEQ ID NO: 1 - DNA, 6030 bp, Homo sapiens
  ref <301>: AUTHORS: Cleary,M.L., Smith,S.D. and Sklar,J.
  ref <302>: TITLE: Cloning and structural analysis of cDNAs for bcl-2 and a hybrid bcl-2/immunoglobulin transcript resulting from the t(14;18) translocation
  ref <303>: JOURNAL: Cell
  ref <304>: VOLUME: 47
  ref <305>: ISSUE: 1
  ref <306>: PAGES: 19-28
  ref <307>: DATE: 1986
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgcttttaatataaagcctgttttgtcttctgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaatcaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaacaatttttaaatataaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 2 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - DNA, 6492 bp, Homo sapiens
  ref <301>: AUTHORS: Saltman,B., Singh,B., Hedvat,C.V., Wreesmann,V.B. and Ghossein,R.
  ref <302>: TITLE: Patterns of expression of cell cycle/apoptosis genes along the spectrum of thyroid carcinoma progression
  ref <303>: JOURNAL: Surgery
  ref <304>: VOLUME: 140
  ref <305>: ISSUE: 6
  ref <306>: PAGES: 899-905
  ref <307>: DATE: 2006
  ref <301>: AUTHORS: Haydee Cottliar,A.S., Noriega,M.F., Narbaitz,M., Rodriguez,A. Slavutsky,I.R.
  ref <302>: TITLE: Association between telomere length and BCL2 gene rearrangements in low- and high-grade non-Hodgkin lymphomas
  ref <303>: JOURNAL: Cancer Genet. Cytogenet.
  ref <304>: VOLUME: 171
  ref <305>: ISSUE: 1
  ref <306>: PAGES: 1-8
  ref <307>: DATE: 2006
  ref <301>: AUTHORS: Maluf,F.C., Cordon-Cardo,C., Verbel,D.A., Satagopan,J.M., Boyle,M.G., Herr,H. Bajorin,D.F.
  ref <302>: TITLE: Assessing interactions between mdm-2, p53, and bcl-2 as prognostic variables in muscle-invasive bladder cancer treated with neo-adjuvant chemotherapy followed by locoregional surgical
  ref <303>: JOURNAL: Ann. Oncol.
  ref <304>: VOLUME: 17
  ref <305>: ISSUE: 11
  ref <306>: PAGES: 1677-1686
  ref <307>: DATE: 2006
  ref <301>: AUTHORS: Porebska,I., Wyrodek,E., Kosacka,M., Adamiak,J., Jankowska,R. Harlozinska-Szmyrka,A.
  ref <302>: TITLE: Apoptotic markers p53, Bcl-2 and Bax in primary lung cancer
  ref <303>: JOURNAL: In Vivo
  ref <304>: VOLUME: 20
  ref <305>: ISSUE: 5
  ref <306>: PAGES: 599-604
  ref <307>: DATE: 2006
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 4 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 5 - DNA, 1207 bp, Homo sapiens
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaggccacaggtccgagatgcgggggttggagtgcgggtgggctcctggggcaatgggaggctgtggagccggcgaaataaaatcagagttgttgcta

SEQ ID NO: 6 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG